(19) United States
(12) Patent Application Publication
(10) Pub. No.: US 2010/0178242 A1
(43) Pub. Date: Jul. 15, 2010

(54) LABELLING METHODS

(76) Inventors: Alexander Jackson, Amersham (GB); Rajiv Bhalla, Amersham (GB)

Correspondence Address: GE HEALTHCARE BIO-SCIENCES CORP.;PATENT DEPARTMENT, 101 CARNEGIE CENTER, PRINCETON, NJ, 08540, US

(21) Appl. No.: 12/663,582

(22) PCT Filed: Jun. 18, 2008
(86) PCT No.: PCT/EP2008/057659

§ 371 Date: Dec. 8, 2009

Related U.S. Application Data
(60) Provisional application No. 60/945,118, filed on Jun. 20, 2007.

(51) Int. Cl. A61K 51/02; A61K 51/04; A61K 51/08; A61K 51/10; C12N 7/00; C12N 5/00; C12N 1/20; C07K 5/12; C07K 5/08; C07K 16/00; C07K 14/575; C07H 21/00; C07H 1/00; C07D 487/08

(52) U.S. Cl. 424/1.17; 424/1.49; 424/1.69; 424/1.73; 424/1.81; 435/235.1; 435/325; 435/252.1; 530/300; 530/317; 530/331; 530/391.1; 530/399; 536/25.32; 536/123.1; 540/472

(57) ABSTRACT

The invention provides a method for radiofluorination of biological vectors such as peptides comprising reaction of a compound of formula (II): or a salt thereof with a source of [¹⁸F]-fluoride, to give a compound of formula (I): or a salt thereof. The method may be effected under mild reaction conditions and offers a more chemoselective labelling approach Novel reagents for use in the radiofluoridation method, and uses of the resultant ¹⁸F-labelled vectors are also provided

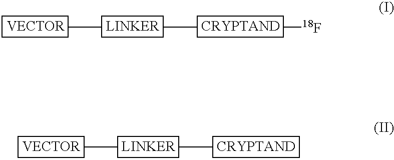

The present invention relates to methods and reagents for [¹⁸F]-fluorination, particularly of biological vectors such as peptides. The resultant ¹⁸F-labelled vectors are useful as radiopharmaceuticals, specifically for use in Positron Emission Tomography (PET).

The application of radiolabelled biological vectors for diagnostic imaging is gaining importance in nuclear medicine. Biologically active molecules which selectively interact with specific cell types are useful for the delivery of radioactivity to target tissues. For example, radiolabelled biological vectors have significant potential for the delivery of radionuclides to tumours, infarcts, and infected tissues for diagnostic imaging, clinical research, and radiotherapy. ¹⁸F, with its half-life of 110 minutes, is the positron-emitting nuclide of choice for many receptor imaging studies. Therefore, ¹⁸F-labelled biological vectors have great clinical potential because of their utility in PET to quantitatively detect and characterise a wide variety of diseases.

One difficulty with certain ¹⁸F-labelled biological vectors is that the existing ¹⁸F-labelling agents are time-consuming to prepare. For example, efficient labelling of peptides and proteins with ¹⁸F is mainly achieved by using suitable prosthetic groups. Several such prosthetic groups have been proposed in the literature, including N-succinimidyl-4-[¹⁸F]fluorobenzoate, m-maleimido-N-(p-[¹⁸F]fluorobenzyl)-benzamide, N-(p-[¹⁸F]fluorophenyl)maleimide, and 4-[¹⁸F]fluorophenacylbromide. Many labelling methods using prosthetic groups give rise to multiple radiolabelled products. For example a peptide containing 3 lysine residues has three amine functions all equally reactive towards the labelled prosthetic group. This approach, often referred to as the “two-step” approach can also be time-consuming as the radiolabelled prosthetic group has to be prepared and then coupled to the biological vector in a second step. Therefore, there still exists a need for ¹⁸F-labelling methodologies which allow rapid, chemoselective introduction of ¹⁸F into biological vectors, particularly into peptides and proteins, under mild conditions to give ¹⁸F-labelled products in high radiochemical yield and purity. Additionally, there is a need for such methodologies which are amenable to automation to facilitate preparation of radiopharmaceuticals in the clinical setting.

Accordingly, the present invention provides a method for radiofluorination comprising reaction of a compound of formula (II):

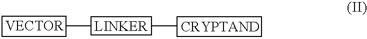

or a salt thereof with a source of [¹⁸F]-fluoride, to give a compound of formula (I):

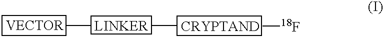

or a salt thereof, followed by the optional steps:
(i) purification of the compound of formula (I); and/or
(ii) formulation of the compound of formula (I).

The present invention provides a more chemoselective approach to radiolabelling where the exact site of introduction of the label is pre-selected during the synthesis of the precursor of formula (II). This methodology is therefore chemoselective and its application is considered generic for a wide range of biological vectors.

As used herein, the term “Vector” means a biomolecule suitable for radiolabelling to form a radiopharmaceutical, such as a peptide, protein, hormone, polysaccaride, oligonucleotide, antibody fragment, cell, bacterium, virus, or small drug-like molecule.

In formulae (I) and (II) and in other aspects of the invention unless specifically stated otherwise, particularly suitable Vectors are selected from peptides, proteins, and small drug-like molecules, and in one aspect of the invention are Vectors which do not need to cross the blood-brain barrier for their biological function.

Suitable peptides for use as a Vector in the invention include somatostatin analogues, such as octreotide, bombesin, vasoactive intestinal peptide, chemotactic peptide analogues, α-melanocyte stimulating hormone, neurotensin, Arg-Gly-Asp peptide, human pro-insulin connecting peptide, insulin, endothelin, angiotensin, bradykinin, endostatin, angiostatin, glutathione, calcitonin, Magainin I and II, luteinizing hormone releasing hormone, gastrins, cholecystochinin, substance P, vasopressin, formyl-norleucyl-leucyl-phenylalanyl-norleucyl-tyrosyl-lysine, Annexin V analogues, Vasoactive Protein-1 (VAP-1) peptides, and caspase peptide substrates. Preferred peptides for use as a Vector in the invention are Arg-Gly-Asp peptide and its analogues, such as those described in WO 01/77415 and WO 03/006491, preferably a peptide comprising the fragment:

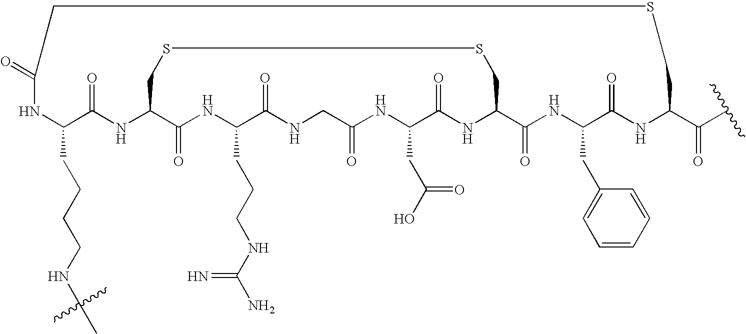

more preferably, the peptide of formula (A):

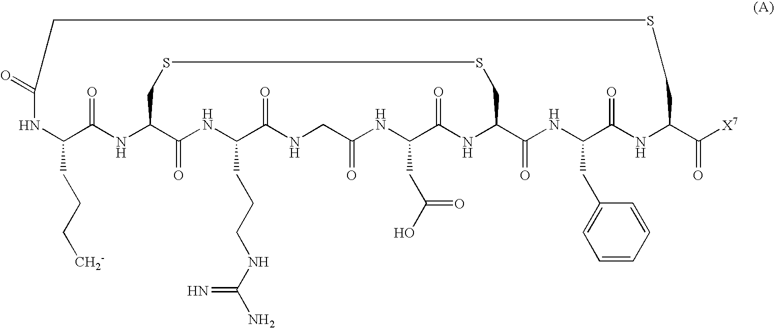

wherein X⁷ is either —NH₂ or

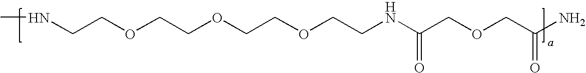

wherein a is an integer of from 1 to 10, preferably a is 1.

In formulae (II) and (I), and in other aspects of the invention, the Linker is a C₁₋₅₀ hydrocarbyl group optionally including 1 to 10 heteroatoms such as oxygen or nitrogen, and may be chosen to provide good in vivo pharmacokinetics, such as favourable excretion characteristics. The term “hydrocarbyl group” means an organic substituent consisting of carbon and hydrogen, such groups may include saturated, unsaturated, or aromatic portions. Suitable Linker groups include alkyl, alkenyl, alkynyl chains, aromatic, polyaromatic, and heteroaromatic rings (for example, triazoles), and polymers comprising ethyleneglycol, amino acid, or carbohydrate subunits any of which may be optionally substituted for example with one or more ether, thiooether, sulphonamide, or amide functionality.

As used herein, the term “Cryptand” means a bi- or poly-cyclic multidentate ligand for the fluoride anion. Suitable Cryptands for binding anions such as fluoride have been reviewed in J. W. Steed, J. L. Atwood in Supramolecular Chemistry (Wiley, New York, 2000), pp 198-249; Supramolecular Chemistry of Anions, Eds. A Bianchi, K Bowmann-James, E. Garcia-Espana (Wiley-VCH, New York, 1997), and P. D. Beer, P. A. Gale, Angew. Chem. 2001, 113, 502; Angew. Chem. Int. Ed. 2001, 40, 486.

Suitable Cryptands used herein include those of formula (C):

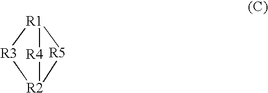

wherein:

R1 and R2 are independently selected from

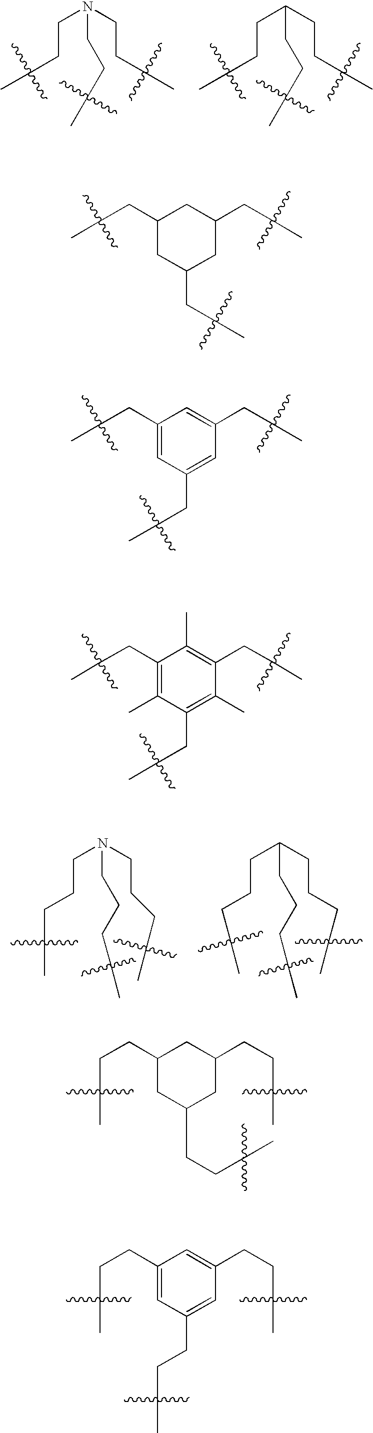

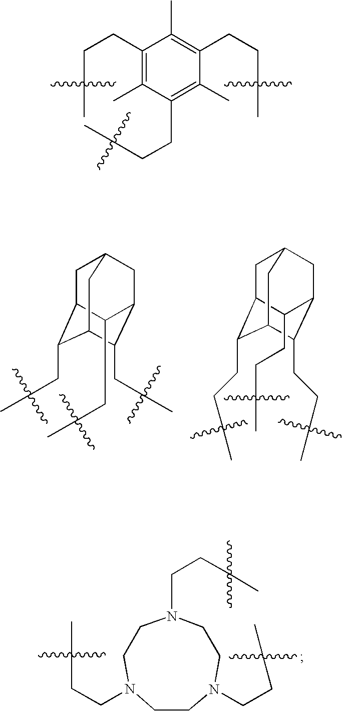

and

R3, R4, and R5 are independently selected from:

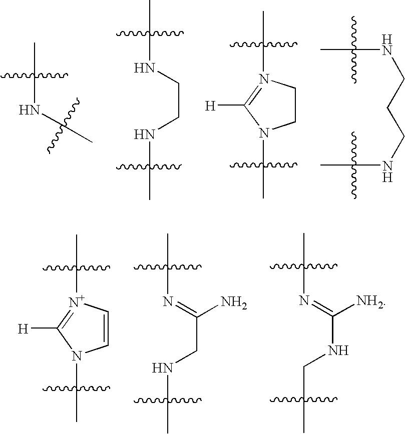

Preferred Cryptands useful in the invention may be selected from:

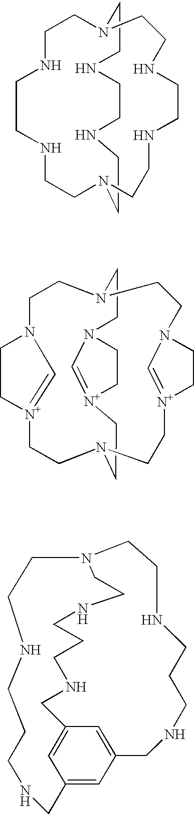

or may be chosen to have desirable properties such as a high binding constant for fluoride, high stability of the fluoride bound complex and high fluoride selectivity over other anions. In one aspect of the invention, the Cryptand bears a positive charge.

In the compounds of formula (I) and (II), the Cryptand is attached to a Linker group. The point of attachment may be a nitrogen or carbon atom in the Cryptand. Thus the point of attachment to the Linker “L” may be in group R1 or R2:

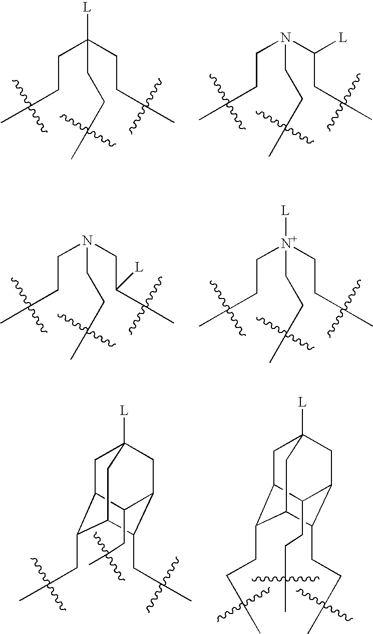

### or in R3, R4, or R5:

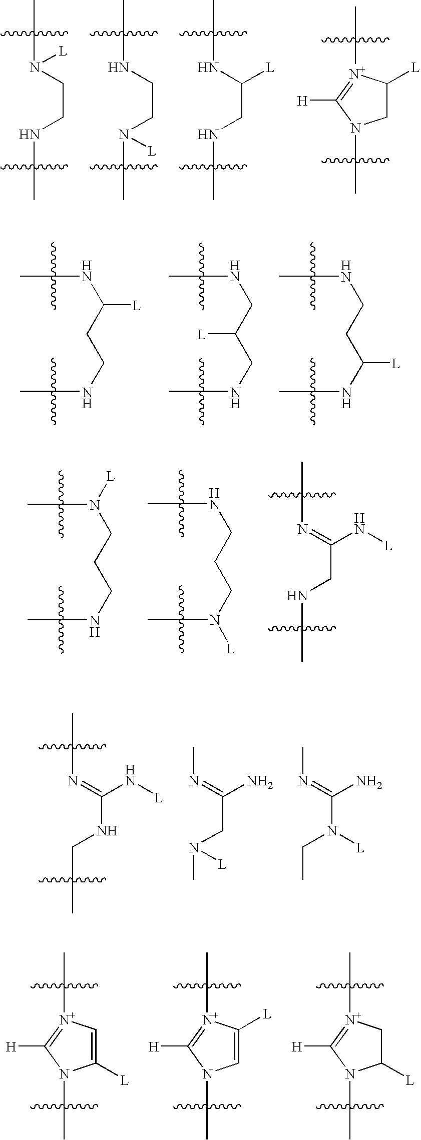

Suitable salts according to the invention include (i) physiologically acceptable acid addition salts such as those derived from mineral acids, for example hydrochloric, hydrobromic, phosphoric, metaphosphoric, nitric and sulphuric acids, and those derived from organic acids, for example tartaric, trifluoroacetic, citric, malic, lactic, fumaric, benzoic, glycolic, gluconic, succinic, methanesulphonic, and para-toluenesulphonic acids; and (ii) physiologically acceptable base salts such as ammonium salts, alkali metal salts (for example those of sodium and potassium), alkaline earth metal salts (for example those of calcium and magnesium), salts with organic bases such as triethanolamine, N-methyl-D-glucamine, piperidine, pyridine, piperazine, and morpholine, and salts with amino acids such as arginine and lysine.

As used herein, the term “source of [¹⁸F]-fluoride” means a reagent capable of delivering [¹⁸F]-fluoride in reactive form to the reaction mixture. [¹⁸F]fluoride is conveniently prepared in a cyclotron from ¹⁸O-enriched water using the (p,n)-nuclear reaction, (Guillaume et al, Appl. Radiat. Isot. 42 (1991) 749-762). For example, the source of [¹⁸F]-fluoride may be [¹⁸F]-fluoride in target water from a cyclotron, or an [¹⁸F]-fluoride salt prepared from the target water such as: [¹⁸F]-sodium fluoride, [¹⁸F]-potassium fluoride, [¹⁸F]-caesium fluoride, [¹⁸F]-tetraalkylammonium fluoride, [¹⁸F]-tetraalkylphosphonium fluoride in a suitable solvent such as acetonitrile, dimethylformamide, dimethylsulphoxide, tetrahydrofuran, dioxan, 1,2-dimethoxyethane, sulpholane, M-methylpyrrolidinone, or aqueous mixtures of any thereof.

The reaction of a compound of formula (II) with a source of [¹⁸F]-fluoride may be effected at non-extreme temperature, such as 10° C. to 50° C., and most preferably at ambient temperature and in a suitable solvent such as those listed above as solvents for the “source of [¹⁸F]-fluoride” or alternatively as a solid supported reaction as described below. The ability to incorporate [¹⁸F]-fluoride into a biological vector at ambient temperature is a particular advantage of the invention as many biological vectors are unstable at elevated temperatures. If the Cryptand in a compound of formula (II) does not have a fixed positive charge, the reaction with a source of [¹⁸F]-fluoride is suitably performed at a pH of below 5, which is achieved by addition of acid such as hydrochloric or sulphuric acid.

Following preparation of a compound of formula (I), a purification step (i) may be required which may comprise, for example, removal of excess [¹⁸F]-fluoride, removal of solvent, and/or separation from unreacted compound of formula (II). Excess [¹⁸F]-fluoride may be removed from a solution of the compound of formula (I) by conventional techniques such as ion-exchange chromatography (for example using BIO-RAD AG 1-X8 or Waters QMA) or solid-phase extraction (for example, using alumina). Excess solvents may be removed by conventional techniques such as evaporation at elevated temperature in vacuo or by passing a stream of inert gas (for example, nitrogen or argon) over the solution. Alternatively, the compound of formula (I) may be trapped on a solid-phase, for example a cartridge of reverse-phase absorbant for example a C₅₋₁₈ derivatized silica, whilst the unwanted excess reagents and by-products are eluted, and then the compound of formula (I) may be eluted from the solid-phase in purified form. Separation of a compound of formula (I) from unreacted compound of formula (II) may be effected by conventional techniques, for example using solid-phase extraction on an anionic solid-phase (for example, a macroporous sulphonated polystyrene resin) exploiting the reduced charge, and hence change in affinity caused by binding of [¹⁸F]-fluoride to the compound of formula (II).

In one embodiment, the compounds of formulae (II) may be covalently bound via the Vector to a solid support, such as polymer beads or coatings, for example, a trityl or chlorotrityl resin. In this aspect, the excess reagents and by-products of the radio-fluorination reaction may be separated from the polymer-bound product by washing. Cleavage of the compound of formula (II) from the solid support may be effected by conventional techniques of solid phase chemistry, for example as described in Florencio Zaragoza Dorwald “Organic Synthesis on Solid Phase; Supports, Linker, Reactions”, Wiley-VCH (2000). This approach may be particularly suitable for automated production of the compounds of formula (I) in which the Vector is a peptide or protein.

Following preparation of a compound of formula (I) or a salt thereof, it may be appropriate to formulate it as a radiopharmaceutical, ready for administration to a subject. Such formulation step (ii) may comprise preparation of an aqueous solution of the compound of formula (I) or a salt thereof by dissolving in sterile isotonic saline which may contain up to 10% of a suitable organic solvent such as ethanol, or a suitable buffered solution such as a phosphate buffer. Other additives such as stabilizers, for example ascorbic acid may be added to the formulation.

Compounds of formula (II) may be prepared by reacting a compound of formula (III):

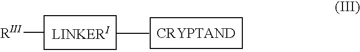

with a compound of formula (IV):

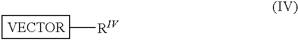

wherein the Vector and Cryptand are as defined above, Linker′ is a portion of the Linker as defined above, and R^{III} and R^{IV} are reactive groups capable of covalent bonding to each other so as to complete formation of the Linker. Suitably, one of R^{III} and R^{IV} is an amine and the other is a carboxylic acid or an activated carboxylic ester, isocyanate or isothiocyanate such that the compounds of formulae (III) and (IV) may be joined by simple amine reaction. Suitable activated carboxylic esters include the N-hydroxysuccinimidyl and N-hydroxysulfosuccinimidyl esters:

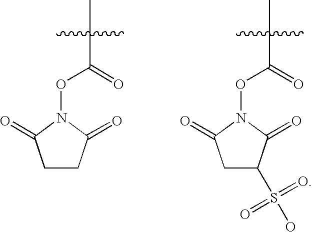

Alternatively one of R^{III} and R^{IV} may be a thiol and the other a group reactive towards a thiol, such as a maleimide or an α-halocarbonyl.

As would be apparent to the person skilled in the art, it may also be desirable for the Cryptand in the Compound of formula (III) to have protection groups on any exposed functional groups e.g. amino groups to prevent or reduce side-reactions during conversion to a Compound of formula (II). In these cases the protection group will be chosen from those commonly used for the functional group in question e.g tert-butylcarbamate for an amine. Other suitable protecting groups may be found in Protecting Groups in Organic Synthesis, Theodora W. Greene and Peter G. M. Wuts, published by John Wiley & Sons Inc. which further describes methods for incorporating and removing such protecting groups.

Certain compounds of formula (II) may be prepared by reacting a compound of formula (III) wherein R^{III} is either an amino or carboxylic acid group with a compound of formula (IV) wherein R^{IV} is either a carboxylic acid or amine group respectively. In these cases a compound of formula (II) may be coupled with a compound of formula (IV) optionally using in situ activating agents such as 2-(1H-benzotriazole-1-yl)-1,1,3,3-tetramethyluronium hexafluorophosphate (HBTU) or N-[(dimethylamino)-1H-1,2,3-triazolo[4,5-b]pyridin-1-ylmethylene]-N-methylmethanamonium hexafluorophosphate N-oxide (HATU). Standard conditions will be used e.g. dimethylformamide (DMF) solution and a base e.g. triethylamine or diisopropylethylamine. Alternatively where R^{IV} in the compound of formula (IV) is a thiol group, this may be reacted with a compound (III) in which R^{III} is a thiol reactive group such as a maleimide or an α-halocarbonyl. This reaction may be performed in a pH buffered solution or an organic solvent. The product compound having the formula (II) might be purified by preparative high performance liquid chromatography.

Compounds of formula (II) wherein the Vector is a peptide or protein may be prepared by standard methods of peptide synthesis, for example, solid-phase peptide synthesis, for example, as described in Atherton, E. and Sheppard, R. C.; “Solid Phase Synthesis”; IRL Press: Oxford, 1989. Incorporation of the Linker and Cryptand in a compound of formula (II) may be achieved by reaction of the N or C-terminus of the peptide or with some other functional group contained within the peptide sequence, modification of which does not affect the binding characteristics of the Vector. The Compound of formula (III) as defined above, is preferably introduced by formation of a stable amide bond formed by reaction of a peptide amine function (R^{IV}) with a compound of formula (III) in which R^{III} is an activated acid or alternatively by reaction of a peptide acid function (R^{IV}) with a compound of formula (III) in which R^{III} is an amine, and in either case the compound of formula (III) may be introduced either during or following the peptide synthesis, for example, solid-phase peptide synthesis. When either of R^{III} or R^{IV} is an acid the reaction of compounds of formulae (III) and (IV) may be effected using in situ activating agents such as 2-(1H-benzotriazole-1-yl)-1,1,3,3-tetramethyluronium hexafluorophosphate (HBTU) or N-[(dimethylamino)-1H-1,2,3-triazolo[4,5-b]pyridin-1-ylmethylene]-N-methylmethanammonium hexafluorophosphate N-oxide (HATU). An embodiment of this particular aspect of the invention is shown in Scheme 1.

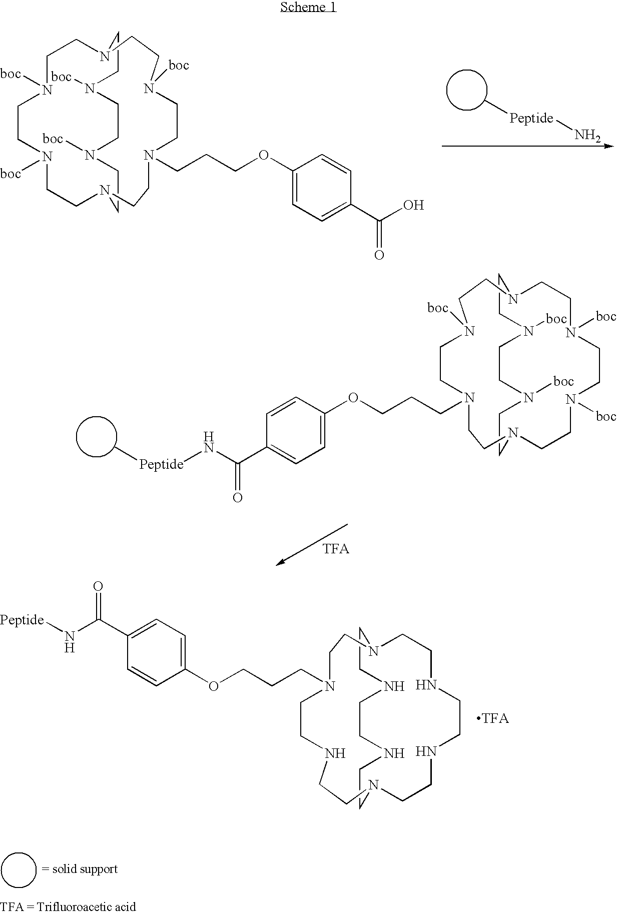

The Cryptands may be synthesised as described in US20040267009 A1, Bernard Dietrich, Jean-Marie Lehn, Jean Guilhem and Claudine Pascard, Tetrahedron Letters, 1989, Vol. 30, No. 31, pp 4125-4128, Paul H. Smith et al, J. Org. Chem., 1993, 58, 7939-7941, Jonathan W. Steed et al, 2004, Journal of the American Chemical Society, 126, 12395-12402, Bing-guang Zhang et al, Chem. Comm., 2004, 2206-2207.

The synthesis of a Compound of formula (III) may be achieved as described in the above references for the underivatized Cryptands with modifications to the starting materials or by subsequent chemistry, for example, by alkylation of a secondary amine group of the Cryptand as illustrated in the Examples below. Compounds of formula (III) may also be prepared as shown in Schemes 2 to 5 in which L and R′″ are as defined above for the Compound of formula (III).

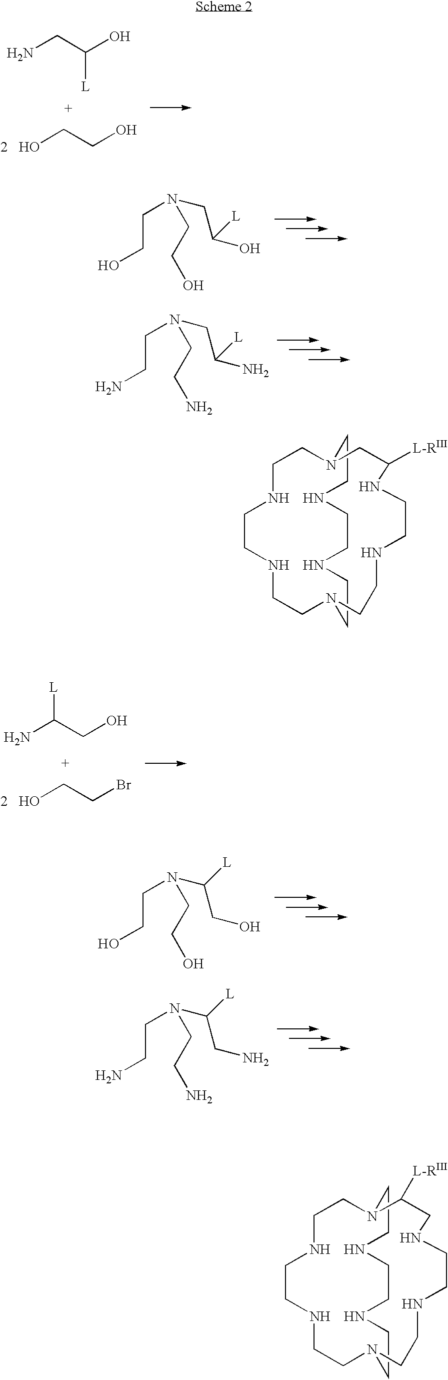

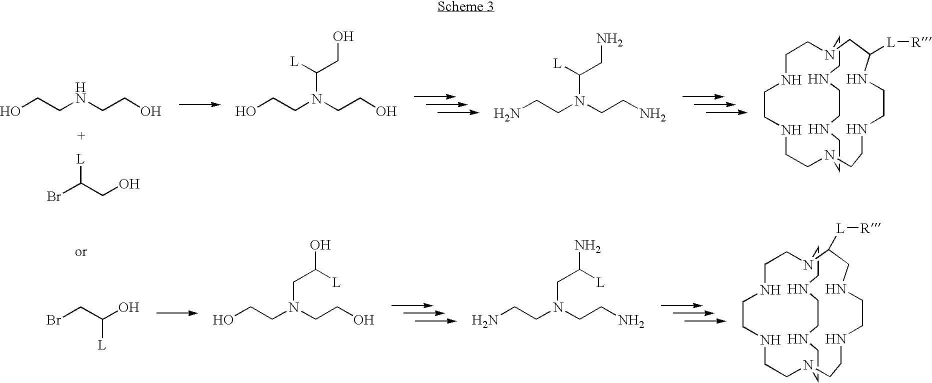

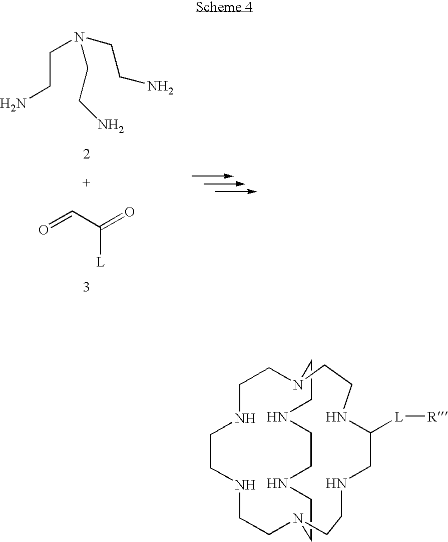

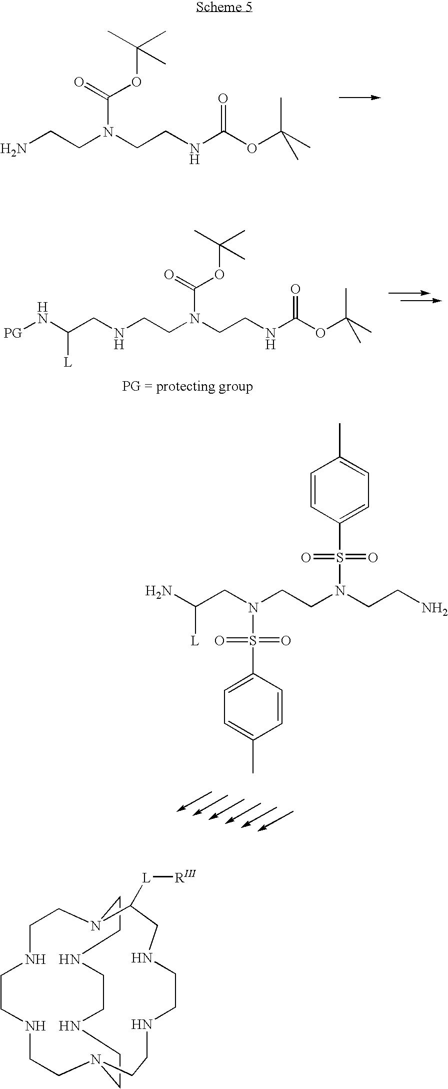

As a further aspect of the invention, there is provided a compound of formula (I) or a salt thereof, as defined above. These compounds having utility as PET tracers. Compounds of formula (I) in which the Vector is a peptide suitably Arg-Gly-Asp peptide or its analogues are preferred, such as the peptides described in WO 01/77145 and WO 03/006491. Particularly preferred peptides in this aspect of the invention are those of formula (A) as defined above for the compounds of formula (I).

The compounds of formula (I) or a salt thereof may be administered to patients for PET imaging in amounts sufficient to yield the desired signal, typical radionuclide dosages of 0.01 to 100 mCi, preferably 0.1 to 50 mCi will normally be sufficient per 70 kg bodyweight, though the exact dose will be dependent on the imaging method being performed and on the composition of the compound of formula (I) or salt thereof.

The compounds of formula (I) or a salt thereof may therefore be formulated as a radiopharmaceutical for administration using physiologically acceptable carriers or excipients in a manner fully within the skill of the art. For example, a compound of formula (I) or a salt thereof, optionally with the addition of one or more pharmaceutically acceptable excipients, may be suspended or dissolved in an aqueous medium, with the resulting solution or suspension then being sterilized. Such radiopharmaceuticals form a further aspect of the invention.

Viewed from a further aspect the invention provides the a compound of formula (I) or a salt thereof as defined above for use in medicine, more particularly in a method of in vivo imaging, suitably PET, said method involving administration of said compound to a human or animal body and generation of an image of at least part of said body.

Viewed from a still further aspect the invention provides a method of generating an image of a human or animal body involving administering a radiopharmaceutical to said body, e.g. into the vascular system and generating an image of at least a part of said body to which said radiopharmaceutical has distributed using PET, wherein said radiopharmaceutical comprises a compound of formula (I) or a salt thereof as defined above. In a further aspect, there is provided a method for in vivo imaging, suitably PET imaging, of a body, preferably a human body, to which body a radiopharmaceutical comprising a compound of formula (I) or a salt thereof as defined above has been pre-administered, wherein the method comprises detecting the uptake of said radiopharmaceutical by an in vivo imaging technique, suitably PET.

In a further aspect, the present invention provides a compound of formula (II) or a salt thereof as defined above, having use as a radiolabelling precursor.

In another aspect, the present invention provides novel synthetic intermediates of formula (III), useful for functionalising Vectors ready for radiofluoridation, for example by the methods described above. Accordingly, there is provided a compound of formula (III):

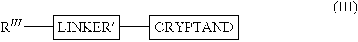

wherein R^{III} is as defined above and is preferably selected from amine, carboxylic acid, activated carboxylic ester, isocyanate, isothiocyanate, thiol, maleimide, or α-halocarbonyl, and the Linker′ and Cryptand are as defined above.

Preferred compounds of formula (III) include:

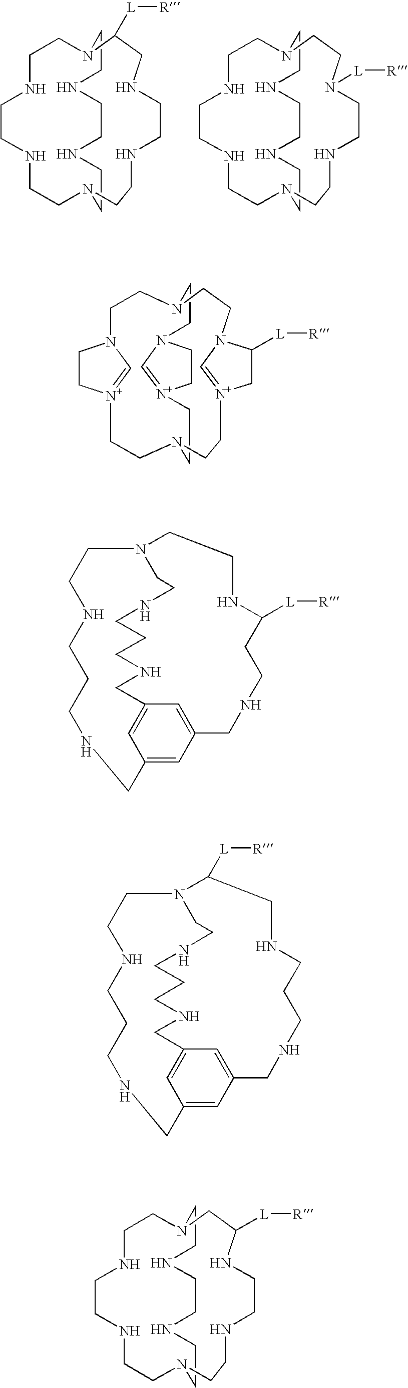

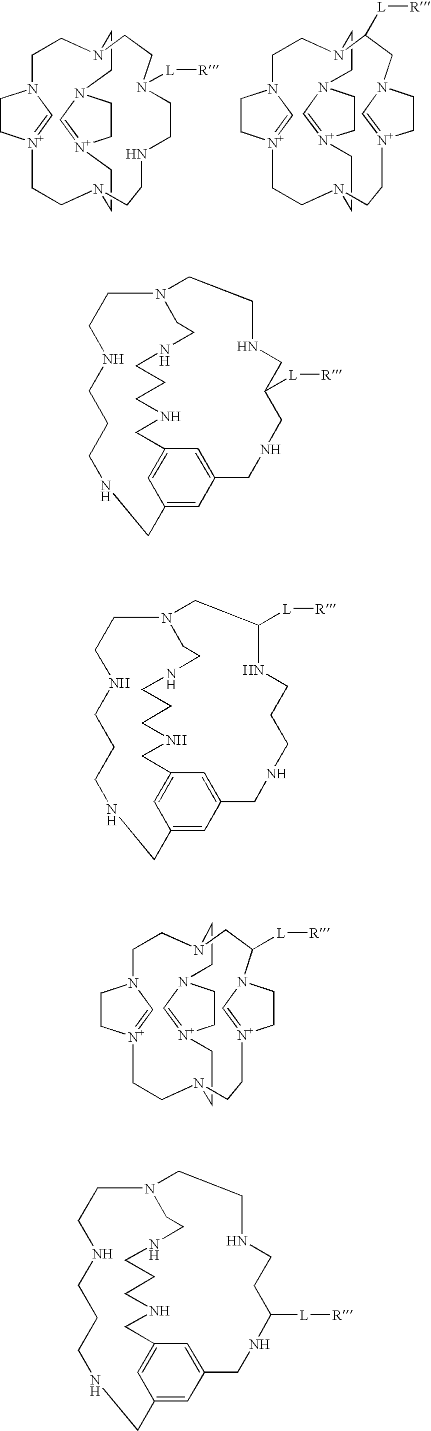

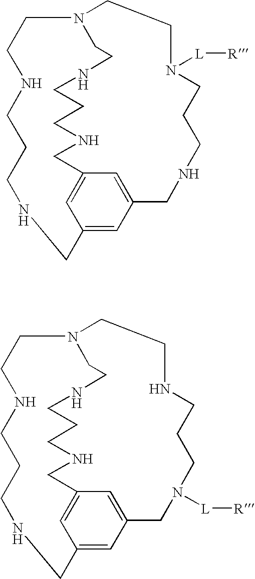

wherein L is a Linker′ as defined above, and R^{III} is a reactive group as defined above, and is preferably selected from amine, carboxylic acid, activated carboxylic ester, isocyanate, isothiocyanate, thiol, maleimide, or α-halocarbonyl.

More preferred compounds of formula (III) include:

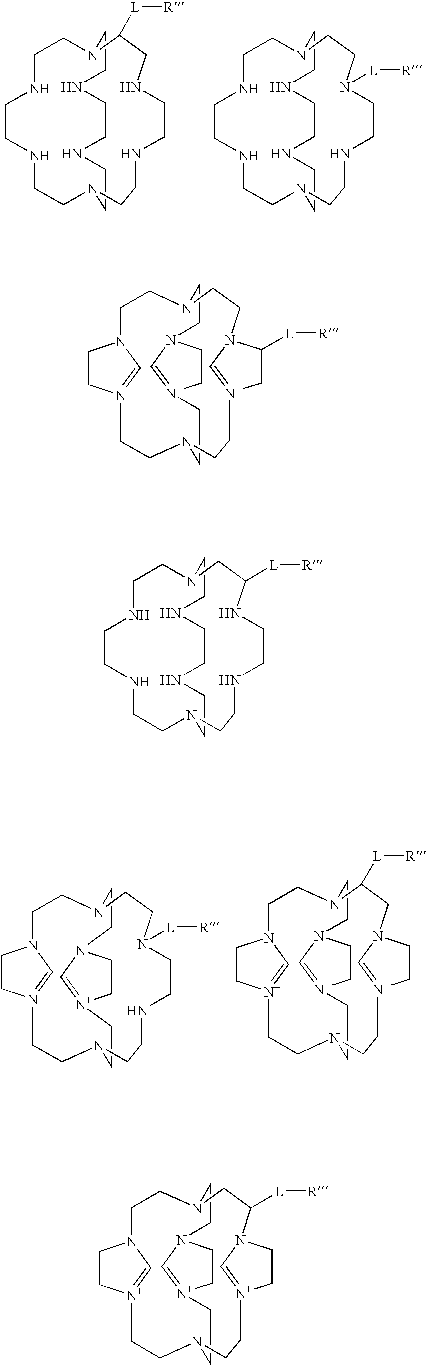

wherein L is a Linker′ as defined above, and is a reactive group as defined above, and is preferably selected from amine, carboxylic acid, activated carboxylic ester, isocyanate, isothiocyanate, thiol, maleimide, or α-halocarbonyl.

In a further aspect of the invention, there is provided a compound of formula (V):

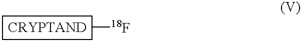

or a salt thereof, wherein the Cryptand is as defined above, for use in medicine, for example as perfusion imaging agents.

Preferred compounds of formula (V) for this purpose comprise a preferred Cryptand as described above. For this use, the Compound of formula (V) or a salt thereof is suitably formulated as a radiopharmaceutical as described above for the Compounds of formula (I).

In the alternative, there is provided a method of imaging which comprises administration to a subject of a detectable amount of a compound of formula (V) or a salt thereof as defined above, and imaging the subject using PET. Methods for perfusion imaging using PET are described in Swaiger, J. Nucl. Med. (1994) 693-8 and the references therein.

In some circumstances, it may be desirable to prepare a prosthetic group for radiofluoridation of a Vector. Therefore, according to a further aspect of the invention there is provided a compound of formula (VI):

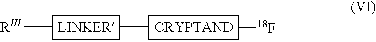

wherein the Linker′ and Cryptand and R^{III} are as defined for a compound of formula (III) above.

According to a further aspect of the invention there is provided a kit for the preparation of a radiofluorinated compound comprising a synthetic intermediate of formula (III), and optionally a compound of formula (IV) as defined above.

In use of the kit, the compound of formula (III), would be reacted with a compound of formula (IV), using methods described above to form the corresponding compound of formula (II) and then reacted with a source of [¹⁸F]-fluoride to form a radiofluorinated Vector of formula (I). Optionally, the compound of formula (I) may be purified and/or formulated as described above.

The invention is illustrated by way of the following examples, in which these abbreviations are used:

PrⁱOH: isopropanol
Et₃N: triethylamine
R.T.: room temperature
MeOH: methanol
(t) BOC: (tertiary) butoxycarbonyl
L: litre
MI: millilitre
hr(s): hour(s)
THF: tetrahydrofuran
HPLC: high performance liquid chromatography
DCM: dichloromethane
LCMS: liquid chromatography mass spectrometry
NMR: nuclear magnetic resonance
TFA: trifluoroacetic acid

### EXAMPLES

### Example 1

### Synthesis of Compound 4

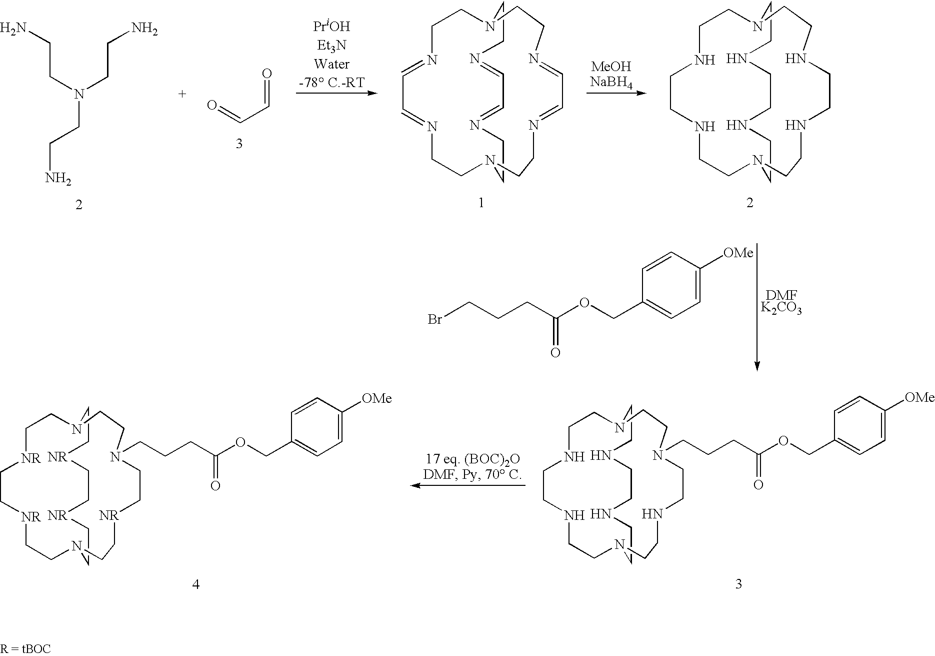

### Example 1(i)

### Synthesis of Compound 1

A 1 L 3-neck round-bottom flask equipped with a mechanical stirrer was charged with 16.7 mL of 98% tripropylamine and 0.33 L of 99% i-PrOH, and cooled to −78° C. in a dry ice-isopropanol bath. To this mixture, solutions of 15.0 g 40% aqueous glyoxal (0.103 mole), diluted to 83 mL with isopropanol, and 10.0 g (0.0.683 moles) of 96% tris-(2-aminoethyl)amine(tren), diluted to 83 mL, were simultaneously added over a period of 2 hrs with vigorous stirring. (Initial concentration of glyoxal=1.24 M; Initial concentration of tren=0.82 M). Then the reaction mixture was allowed to warm up overnight and briefly warmed up to 60° C. to ensure that the formation of compound 2 was complete. It was cooled to room temperature while nitrogen gas was blown over its surface. The solvent was removed under vacuum and chloroform (250 mL) was added. The resulting slurry was filtered through sand and concentrated under vacuum to give an orange solid (5.2 g, 43%).

### Example 1(ii)

### Synthesis of Compound 2

Compound 1 (4 g, 11.2 mmol) was dissolved in methanol ((150 mL (and was cooled in an ice/water bath. Sodium borohydride (8 g, 208 mmol) was added portion wise over 30 minutes. The mixture was left to rise to room temperature with stiffing over 16 hours. The solution was concentrated to dryness under vacuum to give an off white solid. The solid was dissolved in water (100 mL) and was heated to 60° C. for half an hour during which time an oily material formed in the mixture. THF (100 mL) was added and the organic layer was separated. The aqueous layer was extracted again with THF (100 mL). The combined extracts were filtered through a phase separator cartridge and were concentrated to dryness under vacuum. The oily solids were re-dissolved in THF (20 mL) and water (15 mL) was added. The solution was concentrated slowly until a white solid crystallized which was collected by filtration, washed with ice cold water and dried under high vacuum (1.6 g, 38%).

### Example 1(iii)

### Synthesis of Compound 3

Compound 2 (0.1 g, 0.270 mmol) was dissolved in dry DMF (5 mL) and potassium carbonate added (1.1 eq. 0.297 mmol, 0.041 g). The alkyl bromide (1.1 eq. 0.297 mmol, 81.7 mg) was added portion wise following the reaction by HPLC-mass spectrometry by taking approximately 0.1 mL volume from the reaction and diluting with 1:1 0.1% formic acid in water:acetonitrile (10 mL). The reaction was stirred at room temperature for 16 hours. A further 0.25 equivalents of the alkyl bromide was added and the reaction stirred for a further 16 hours. The reaction mixture was concentrated to dryness under vacuum. This was used in the next step without further purification.

### Example 1(iv)

### Synthesis of Compound 4

Crude compound 3 was dissolved in dry DMF (20 mL) and pyridine (2 mL) was added followed by di-tert-butylcarbonate (1 g, 4.58 mmol, 17 eq.). The mixture was heated at 70° C. under nitrogen for 16 hours. The crude product was analysed by thin layer chromatography (silica gel plates eluting with 10% methanol/DCM) and by LCMS. Thin layer chromatography showed two major spots having Rf values of 0.2 and 0.5 and some minor spots. The mixture was purified by flash column chromatography on silica gel eluting with 100% petrol 40-60 to 100% ethyl acetate. The second major peak was shown to be the desired penta-BOC product by NMR and LCMS (50 mg).

### Example 2

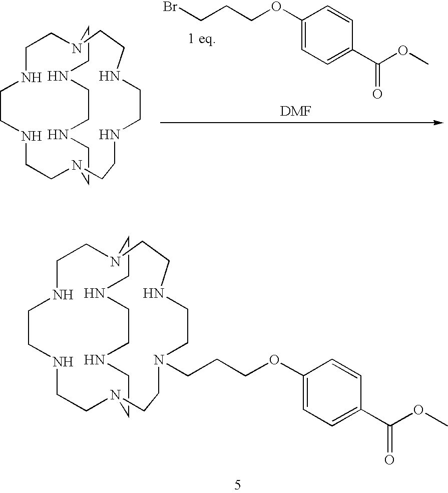

### Example 2(i)

### Synthesis of Compound 5

Compound 2 (0.1 g, 0.270 mmol) was dissolved in dry DMF (2 mL (and a solution of the alkyl bromide (1.1 eq. 0.297 mmol, 81.07 mg) in dry DMF (1 mL) was added over 5 minutes. The solution was stirred at room temperature for 16 hours. The DMF was removed under reduced pressure and white solids dissolved in an minimum volume of water/methanol (1:1). Preparative HPLC (Phenomenex luna C18(2) 150×21.2, acetonitrile/water 5% to 70% over 10 minutes) gave a major peak having tᵣ of 8-8.5 minutes which was freeze dried giving an white solid (15 mg). NMR and LCMS confirmed the structure.

### Example 2(ii)

### Fluoride Binding Studies with [¹⁹F]-Fluoride

Compound 5 (1 mg) in water (0.1 mL) acidified to pH 1 with 1N HCl and an aqueous solution of potassium fluoride (0.1-1 eq) was added at RT. The solutions were analysed by reversed phase HPLC (1% TFA/water, 1% TFA MeCN gradient on Luna C5 150×4.6 mm, detecting at 254 nm).

### Example 2(iii)

### Fluoride Radiolabelling of Compound 5 with [¹⁸F]-Fluoride

1M HCl (4.5 μL, 4.5 μmol) was added to compound 5 (0.1 mg, 180 nmol) in 50:50 methanol/water (0.2 mL). This acidified solution was added directly to a glass vial containing [¹⁸F]fluoride (98 MBq) in target water (0.05 mL) and left at room temperature for 20 minutes. The reaction was analyzed by reverse phase HPLC (solvent A=0.1% TFA in water; Solvent B=0.1% TFA in MeCN, Luna C5 150×4.6 mm, detecting at 254 nm; Gradient: 0 to 3 minutes (2% B), 3-10 minutes (2 to 70% B), 10 to 13 minutes (70% B); 13 to 16 minutes (70 to 2% B), 16 to 21 minutes (2% B); flow rate 1 mL/minute. [¹⁸F]-5 has a retention time of 10.1 minutes. [¹⁸F]-5 was purified using the same HPLC method with a decay corrected isolated yield of 64%.

What is claimed is:

1. A method for radiofluorination comprising reaction of a compound of formula (II): 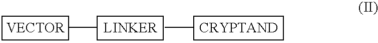
or a salt thereof with a source of [¹⁸F]-fluoride,
to give a compound of formula (I): 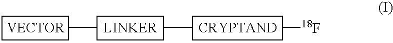
or a salt thereof, followed by the optional steps:
(i) purification of the compound of formula (I); and/or
(ii) formulation of the compound of formula (I); and
wherein said Vector is a biomolecule suitable for radiolabelling; said Linker is a C₁₋₅₀ hydrocarbyl group optionally including 1 to 10 heteroatoms; and said Cryptand is a bi- or poly-cyclic multidentate ligand for a fluoride anion.

2. A method according to claim 1 wherein the Vector is a peptide, protein, hormone, polysaccaride, oligonucleotide, antibody fragment, cell, bacterium, virus, or small drug-like molecule.

3. A method according to claim 1 wherein the Vector is Arg-Gly-Asp peptide or an analogue thereof.

4. A method according to claim 3 wherein the Vector comprises the fragment:

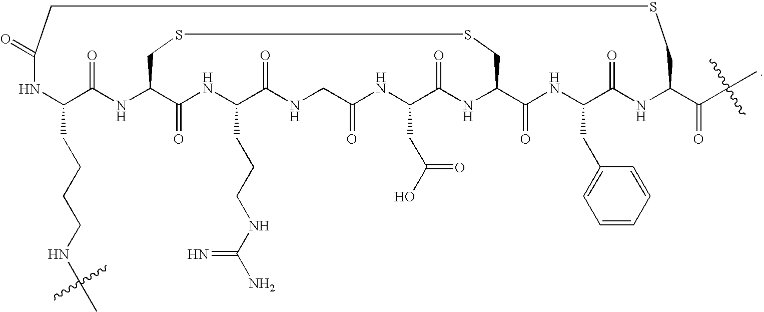

5. A method according to claim 4 wherein the Vector is of formula (A): 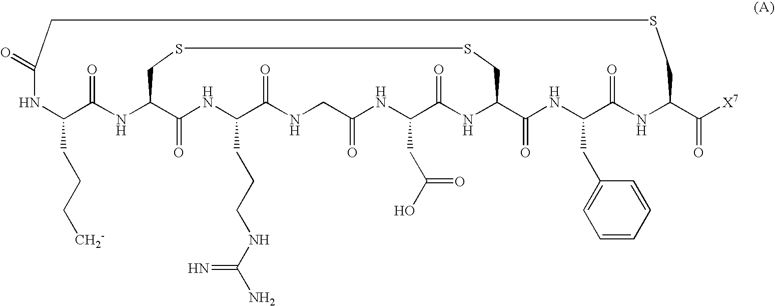
wherein X⁷ is either —NH₂ or 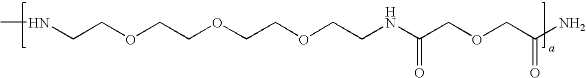
wherein a is an integer of from 1 to 10, preferably a is 1.

6. A method according to claim 1 wherein the Cryptand is of formula (C): 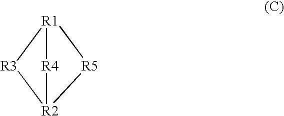
wherein:
R1 and R2 are independently selected from 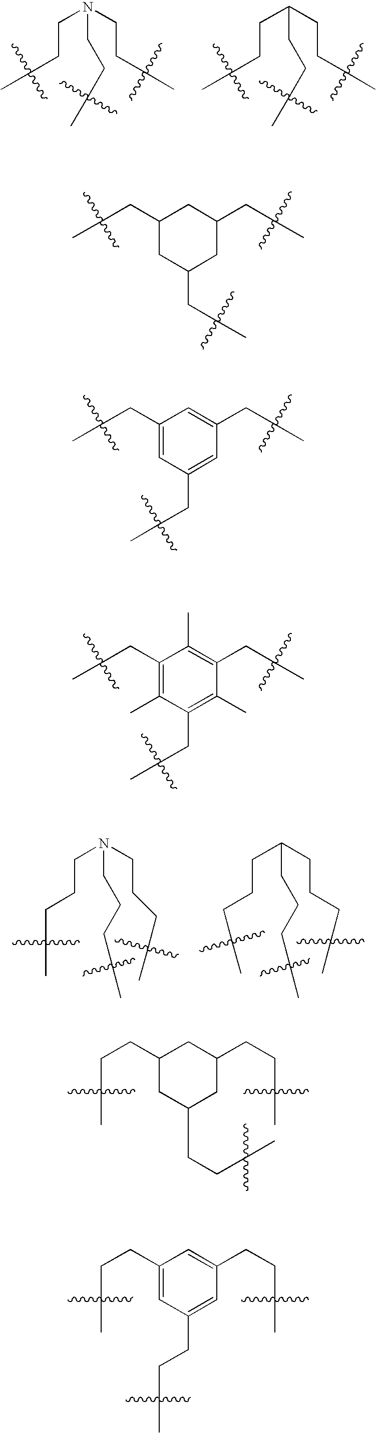 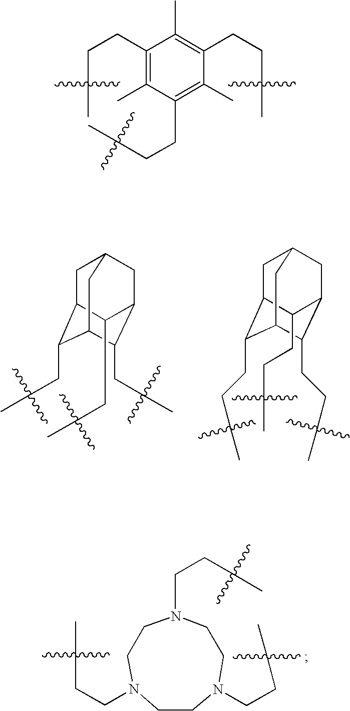
and
R3, R4, and R5 are independently selected from:

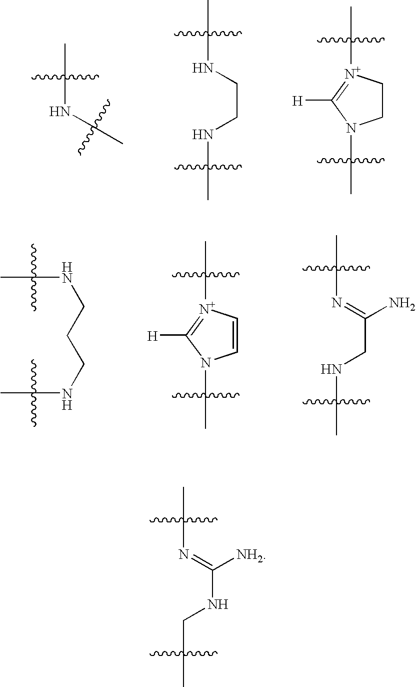

7. A method according to claim 6 wherein the Cryptand is selected from

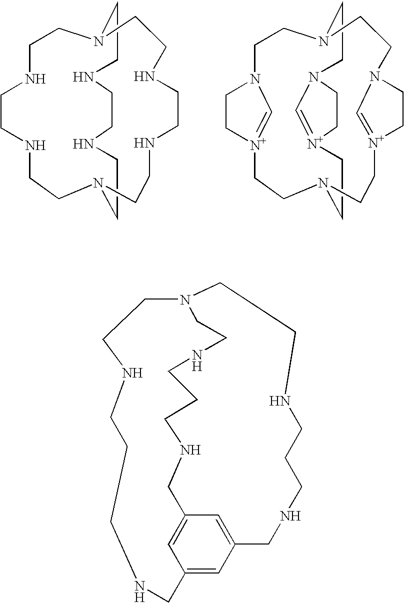

8. A compound of formula (I) or (II) or a salt thereof as defined in claim 1.

9. A radiopharmaceutical formulation comprising a compound of formula (I) or a salt thereof as defined in claim 1 and a physiologically acceptable carrier or excipient.

10. (canceled)

11. A method of generating an image of a human or animal body involving administering a radiopharmaceutical formulation as defined in claim 9 to said body, and generating an image of at least a part of said body to which said radiopharmaceutical has distributed using PET.

12. A compound of formula (III): 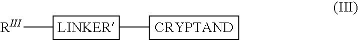
wherein R^{III} is a reactive group suitably selected from amine, carboxylic acid, activated carboxylic ester, isocyanate, isothiocyanate, thiol, maleimide, and α-halocarbonyl; and the Linker′ is a portion of the Linker as defined in claim 1 and the Cryptand is as defined in claim 1.

13. A compound of formula (V): 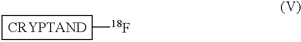
or a salt thereof wherein the Cryptand as defined in claim 1, for use in medicine, for example as perfusion imaging agents.

14. A method of imaging which comprises administration to a subject of a detectable amount of a compound of formula (V) or a salt thereof as defined in claim 13, and imaging the subject using PET.

15. A compound of formula (VI): 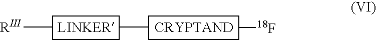
wherein R^{III} is a reactive group suitably selected from amine, carboxylic acid, activated carboxylic ester, isocyanate, isothiocyanate, thiol, maleimide, and α-halocarbonyl; and the Linker′ is a portion of the Linker as defined in claim 1 and the Cryptand is as defined in claim 1.
